(19) United States
(12) Patent Application Publication
(10) Pub. No.: US 2026/0279488 A1
(43) Pub. Date: Sep. 17, 2026

(54) SYSTEMS AND METHODS FOR CLASSIFIYING UNKNOWN SAMPLES INTO KNOWN GENOTYPES

(71) Applicant: Roche Sequencing Solutions, Inc., Pleasnanton, CA (US)

(72) Inventor: Ronald Kurnik, Foster City, CA (US)

(21) Appl. No.: 19/676,011

(22) Filed: May 13, 2026

Related U.S. Application Data
(63) Continuation of application No. PCT/EP2024/082084, filed on Nov. 12, 2024.
(60) Provisional application No. 63/598,250, filed on Nov. 13, 2023.

(51) Int. Cl. G16B 20/00; G16B 40/10

(52) U.S. Cl. CPC G16B 20/00; G16B 40/10

(57) ABSTRACT

The present disclosure relates to classifying genotypes on an assay plate according to predefined standards. As one example, a method includes: obtaining all raw melt curves for an assay plate from a memory; determining melting peak curves for all standards and unknown samples; calculating a median or mean peak curve for each of the standards based on a plurality of replicate melting peak curves of each standard; calculating at least one correlation coefficient between each of the unknown samples and the median standards; comparing the correlation coefficient to a threshold level for each standard; and assigning the unknown sample a genotype of the standard when the unknown sample has a correlation coefficient greater than the threshold level.

### CROSS REFERENCES TO RELATED APPLICATIONS

The present application is a continuation of International Patent Application No. PCT/EP2024/082084, filed Nov. 12, 2024, which application claims benefit of priority to U.S. Provisional Application No. 63/598,250, filed Nov. 13, 2023, each of which is incorporated by reference herein in its entirety.

### TECHNICAL FIELD

The present disclosure relates to methods for classifying genotypes on an assay plate according to predefined standards, and more particularly, to methods for classifying unknown samples into a known genotype.

### BACKGROUND

The polymerase chain reaction (PCR) has become a ubiquitous tool of biomedical research, disease monitoring, and diagnostics. Melting curve analysis has similarly become a common tool used to identify DNA genotypes, often performed after PCR. Melting curve analysis assesses the dissociation characteristics of double strand DNA during heating. In particular, fluorescent dyes bound to double strand DNA typically lose fluorescence as the temperature increases and exhibit a reduction in fluorescence that coincides with the effective dissociation of the DNA. Since different genotypes dissociate at different temperatures, different genotypes thus have melting curves with different profiles. The temperature at which this effective dissociation occurs is often ascertained by identifying peaks of the melting curve. Thus, curves of unknown genotypes that have similar features (such as the peaks) may indicate curves belonging to a known genotype. Accordingly, analysis of the melting curves can be used to identify a genotype.

While methods exist for such genotyping based on melting curve analysis, the previous methods have limitations. For example, existing methods are of limited effectiveness when dealing with a diversity of genotypes, including assays with a large number of genotypes and/or instances where the quality of the melting curves is inconsistent.

### SUMMARY

The present disclosure provides for novel methods for classifying genotypes on an assay plate according to predefined standards. In an aspects, a method incudes obtaining all raw melt curves for an assay plate from a memory. The method also includes determining melting peak curves for all standards and unknown samples. The method also includes calculating a median or mean peak curve for each of the standards based on a plurality of replicate melting peak curves of each standard. The method also includes calculating at least one correlation coefficient between each of the unknown samples and the median standards. The method also includes comparing the correlation coefficient to a threshold level for each standard. The method also includes assigning the unknown sample a genotype of the standard when the unknown sample has a correlation coefficient greater than the threshold level.

In some aspects, the method also includes assigning the unknown sample as an unknown genotype when the unknown sample has a correlation coefficient less than or equal to the threshold level.

In some aspects, the unknown sample is assigned as an unknown genotype when it qualifies as a non-negative curve

In some aspects, determining the melting peak curves includes determining first and second derivatives of each of the raw melt curves curve using a smoothing filter.

In some aspects, the smoothing filter is a Savitzky-Golay technique.

In some aspects, the melting peak curves are based on a plurality of parameters.

In some aspects, the plurality of parameters include a channel number of the curve, temperature values of an input curve, fluorescence values of the input curve, a standard identification of the curve, and a melting temperature of the curve.

In some aspects, the method includes determining a standard curve quality metric to indicate how close each curve within a standard is to a curve representing the median or mean of that standard.

In another aspect, a system includes a memory and a processor coupled to the memory. The processor is configured to obtain all raw melt curves for an assay plate from a memory. The processor is further configured to determine melting peak curves for all standards and unknown samples. The processor is further configured to calculate a median or mean peak curve for each of the standards based on a plurality of replicate melting peak curves of each standard. The processor is further configured to calculate at least one correlation coefficient between each of the unknown samples and the median standards. The processor is further configured to compare the correlation coefficient to a threshold level for each standard. The processor is further configured to assign the unknown sample a genotype of the standard when the unknown sample has a correlation coefficient greater than the threshold level.

### BRIEF DESCRIPTION OF THE DRAWINGS

FIG. 1 is a block diagram illustrating an embodiment an optical system of an analyzer, according to aspects of the present disclosure.

FIG. 2 shows a method for classifying unknown samples into known genotypes, according to aspects of the present disclosure.

FIG. 3 is a block diagram of a computing system in accordance with embodiments of the present disclosure.

FIG. 4 is an illustration of example raw melt curves, according to aspects of the present disclosure.

FIG. 5 illustrates example the median or mean curves of the three different standards, according to aspects of the present disclosure.

FIGS. 6-8 illustrates curves of a different unknown sample, according to aspects of the present disclosure.

### DETAILED DESCRIPTION

In the context of in vitro diagnostic (IVD) assays, it is desirable to determine different genotypes. To that end, and as described above, post-PCR melting steps have been used to determine different genotypes of a given assay. Since the fluorescent dyes bound to double strand DNA lose fluorescence as the temperature increases depending on the characteristics thereof, different genotypes of a given assay will exhibit different fluorescent signal profiles. In order to use melting curves to determine genotypes, typically, the raw melting curve, i.e., the measured fluorescence over the range of temperatures during the melting step, is processed by taking the negative first derivative thereof. This results in curves with a series of peaks. The peaks are usually Gaussian shaped peaks, but can be peaks of other forms, such as Lorentzian, Voigt, Pearson, Compton Edge, or any other types of peaks. Curves which have peaks that resemble each other in shape and frequency, and curves with similar melting temperatures can be grouped together in a single genotype. While existing methods may be able to identify genotypes from a limited number of genotypes, there is still a desire for an automated method for determining genotypes when there is an unknown and/or relatively large number of genotypes for a given assay. For example, a given assay may have anywhere from one to twelve different genotypes, and existing methods are not sufficient to automatically determine genotypes from the melting curves where the genotypes are unknown.

The present disclosure provides for novel methods for classifying genotypes on an assay plate according to predefined standards. The processes described herein include determining melting peak curves for both standard genotypes and unknown samples, calculating median (or mean) values of the individual standard genotypes, evaluating correlation coefficients between the unknown samples and standard genotypes, and when the correlation coefficient exceeds a given threshold value, assigning the unknown sample to a given standard genotype. In some instances, the processes described herein further include outputting the genotype assigned to each unknown sample and melting temperatures of each curve.

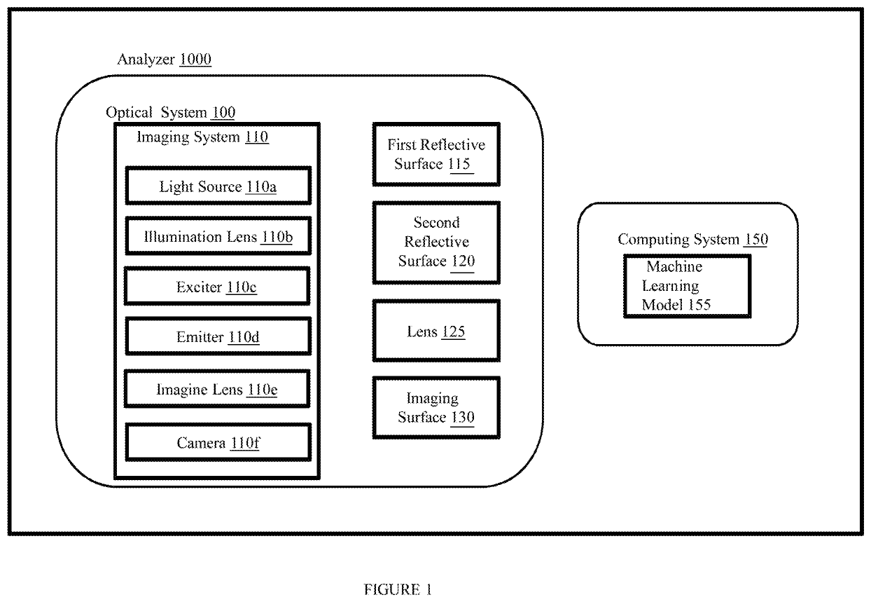

FIG. 1 is a block diagram illustrating an embodiment of an analyzer, according to aspects of the present disclosure. For example, as shown in FIG. 1, an analyzer 1000 includes an optical system 100 and a computing system 150. In some embodiments, the optical system 100 includes an imaging system 110, first and second reflective surfaces 115 and 120, respectively, a lens 125, and an imaging surface 130. In some embodiments, the imaging system 110 may include a light source 110a configured to generate a beam of light, an illumination lens 110b configured to focus the beam of light, and an exciter 110c configured to transmit the focused beam of light onto the first reflective surface 115. In some embodiments, the focused beam of light is reflected off the first reflective surface 115 onto the second reflective surface 120, which in turn is transmitted onto the imaging surface 130 through the lens 125.

In turn, light is reflected from the imagining surface 130 through lens 125 and off of the first and second reflective surfaces 115, 120 onto the imaging system 110. In some embodiments, the imaging system 110 may further include an emitter 110d configured to receive the reflected light from the imaging surface 130, an imaging lens 110e configured to focus the reflected light, and a camera 110f configured to capture the reflected light from the imaging surface 130. In some embodiments, the camera 110f can be used for fluorescence imaging due to its high sensitivity, low noise, and high temporal stability.

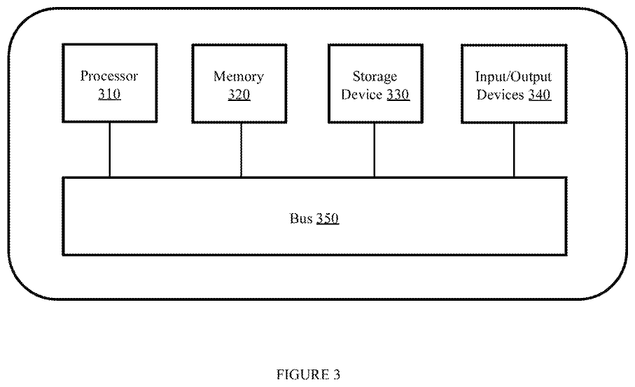

In some embodiments, the computing system 150 may execute one or more processes for classifying genotypes. An example architecture of the computing system 150 is shown in FIG. 3, discussed in greater detail below.

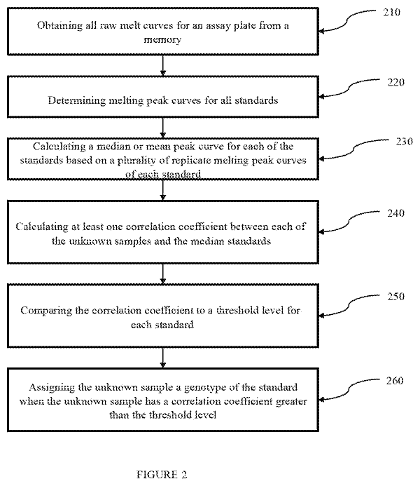

FIG. 2 illustrates a method 200 for classifying genotypes on an assay plate according to predefined standards. The processes described herein include determining, melting peak curves for both standards. In some embodiments, the processes described herein may be performed using a processor, e.g., processor 310 of FIG. 3. At step 210, the method 200 includes obtaining all raw melt curves for an assay plate from a memory. For example, the raw melt curves may be stored on a memory, e.g., memory 320 of FIG. 3, and the processor may obtain the raw melt curves from the memory.

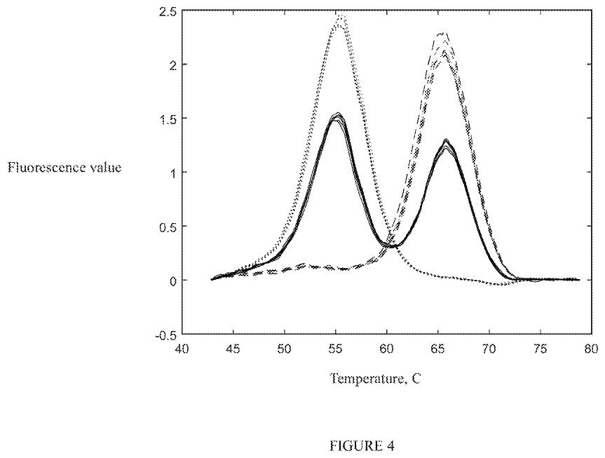

Example raw melt curves are shown in FIG. 4. As can be seen in FIG. 4, the raw melting curves depict the measured fluorescence over a range of temperatures during the melting step after PCR. In this example, the assay includes three known genotypes, e.g., std 1, std 2, and std 3. It should be understood by those of ordinary skill in the art that FIG. 4 illustrates three different standards for illustrative purposes and that more or less standards are contemplated in accordance with aspects of the present disclosure. As shown in FIG. 4, the raw melting curves include Gaussian peaks, and there are groups of similar curves that are similar in the peak height, frequency, and location within the temperature range. While this can be seen visually, it would be burdensome and require extensive manual input to genotype these curves without knowing the number of actual genotypes in the assay. In some embodiments, these raw melting curves may be processed in order to accurately genotype unknown samples in the assay.

At step 220, the method 200 also includes determining melting peak curves for all standards, e.g., known samples, and unknown samples. To achieve this, the processor 310 may determine first and second derivatives of each of the raw melt curves curve using a smoothing filter, which may then be used to calculate the melting temperatures of each curve. In some embodiments, the smoothing filter may be, for example, a Savitzky-Golay technique, although it should be understood that this is merely an example, and that other smoothing filters are further contemplated in accordance with aspects of the present disclosure.

In some embodiments, the melting peak curves may be based on a plurality of parameters. For example, the plurality of parameters may include, but are not limited to, a channel number of the curve, temperature values of an input curve, fluorescence values of the input curve, a standard identification of the curve, and a melting temperature of the curve. FIG. 4 illustrates example curves of three different standards std1, std2, and std 3.

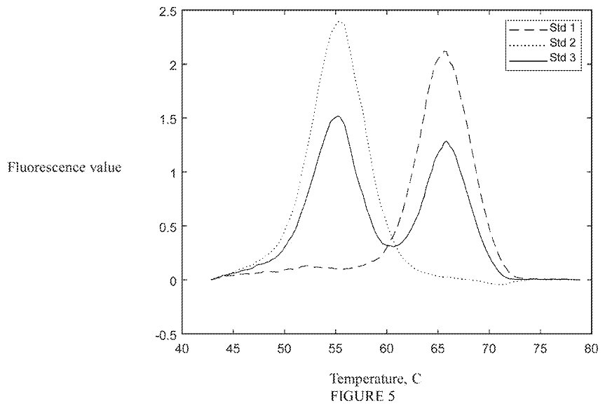

At step 230, the method 200 also includes calculating a median or mean peak curve for each of the standards based on a plurality of replicate melting peak curves of each standard. Calculating the median or peak curve for each standard may be performed using the processor 310. FIG. 5 illustrates example the median or mean curves of the three different standards. In some embodiments, a standard curve quality metric may be determined to indicate how close each curve within a standard is to a curve representing the median or mean of that standard. For example, within each standard group, e.g., each cluster of non-negative curves, a mean and standard deviation of positive fluorescence values may be determined, and this array may be ordered in descending values of the mean. Additionally, a first half of this array may be used to calculate the standard quality metric as shown in equation (2):

$$qMetric = \{ {qMetric}^{*} = round \{ 100 [ 1 - mean ( \frac{stdev ( grp\_curves )}{mean ( grp\_curves )} ) ] \} , {qMetric}^{*} \geq 0.1 , else \qquad ( 2 )$$

This metric may be a number, e.g., a percentage, indicating how close each curve within a standard is to a curve representing the median of that standard.

At step 240, the method 200 also includes calculating at least one correlation coefficient between each of the unknown samples and the median standards. Calculating the at least one correlation coefficient may be performed using the processor 310. In some embodiments, the correlation coefficient between two vectors x and y may be calculated as shown in Equation (1):

$${r}_{x y} = \frac{{\sum }_{i} {x}_{i} {y}_{i} - n \cdot \underline{x} \cdot \underline{y}}{\sqrt{{\sum }_{i} {x}_{i}^{2} - {\underline{x}}^{2}} \sqrt{{\sum }_{i} {y}_{i}^{2} - {\underline{y}}^{2}}} \qquad ( 1 )$$

In some embodiments, n may be a length of both x and y, i.e., they have the same length, and x and y are the mean values of x and y.

At step 250, the method 200 also includes comparing the correlation coefficient to a threshold level for each standard. Comparing the correlation coefficient to a threshold level for each standard may be performed using the processor 310. For example, the threshold level may be 0.95 out of 1.0. It should be understood by those of ordinary skill in the art this is merely an example threshold level and that other threshold levels are further contemplated in accordance with aspects of the present disclosure. For example, the threshold level may be higher, e.g., 0.98, or lower, e.g., 0.93.

At step 260, the method 200 may include assigning the unknown sample a genotype of the standard when the unknown sample has a correlation coefficient greater than the threshold level. In contrast, when the unknown sample has a correlation coefficient less than or equal to the threshold level, the unknown sample may be assigned as an unknown genotype. Assigning the unknown sample to a given genotype or an unknown genotype may be performed by the processor 310. In some instances, an unknown sample may be assigned as an unknown genotype when it qualifies as a negative curve. An unknown sample may designated as negative when a maximum signal of the unknown sample is less than a product of the threshold multiplied by the maximum signal in the assay. It should be understood by a person of ordinary skill in the art that negative standards cannot be used to identify negative curves, as the correlation coefficients would be very small, and negative standards are outside the scope of the present disclosure. In some embodiments, when there is a plurality of negative unknown samples, the plurality of negative unknown samples may be merged with one another.

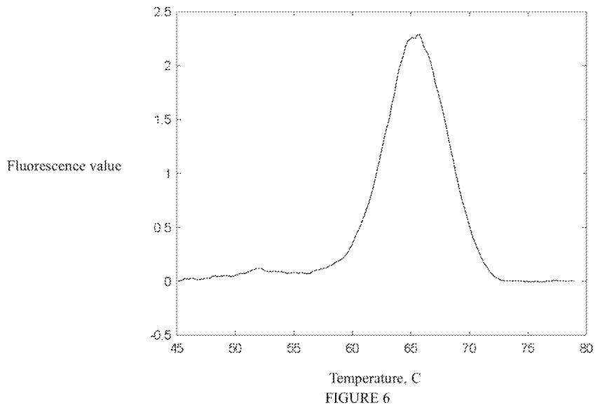

FIG. 6 illustrates a curve of a first unknown sample. In this example, the correlation coefficient of the first unknown sample with respect to the plurality of standards, e.g., std 1, std 2 and std 3, is [0.9985, −0.2717, 0.4971], respectively. By comparing the correlation coefficients to the threshold level, e.g., 0.95, this example signifies that the first unknown sample should be assigned to the standard std 1.

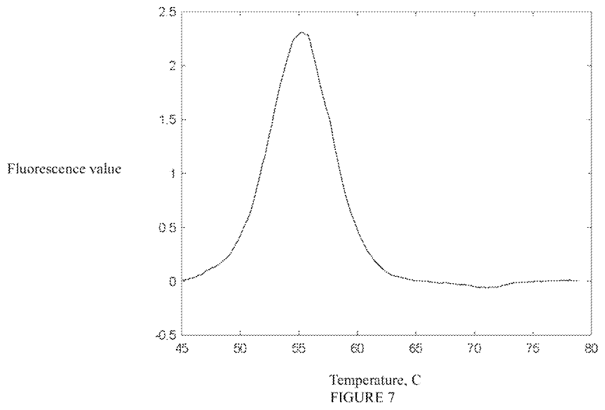

FIG. 7 illustrates a curve of a second unknown sample. In this example, the correlation coefficient of the second unknown sample with respect to the plurality of standards, e.g., std 1, std 2 and std 3, is [−0.2730, 0.9993, 0.6820], respectively. By comparing the correlation coefficients to the threshold level, e.g., 0.95, this example signifies that the second unknown sample should be assigned to the standard std 2.

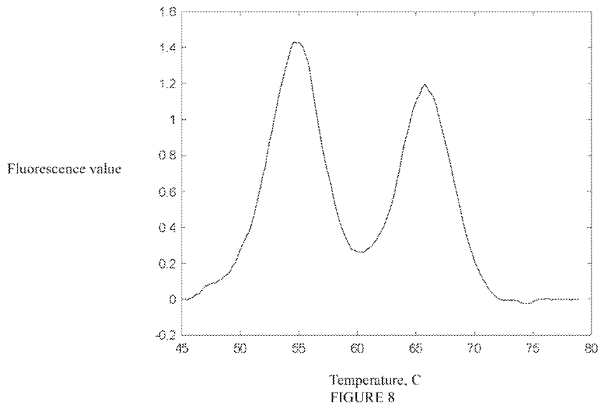

FIG. 8 illustrates a curve of a third unknown sample. In this example, the correlation coefficient of the third unknown sample with respect to the plurality of standards, e.g., std 1, std 2 and std 3, is [0.4969, 0.6820, 0.9973], respectively. By comparing the correlation coefficients to the threshold level, e.g., 0.95, this example signifies that the second unknown sample should be assigned to the standard std 3.

In some embodiments, the genotype assigned to each unknown sample and melting temperatures of each curve may be output and displayed to a user. For example, the output may be displayed on an input/output devices 340 of FIG. 3.

FIG. 3 depicts a block diagram illustrating an example of computing system 300, in accordance with some example embodiments. In some embodiments, the computing system 300 may be to implement the method 200 and/or any components therein.

As shown in FIG. 3, computing system 300 can include a processor 310, a memory 320, a storage device 330, and input/output devices 340. Processor 310, memory 320, storage device 330, and input/output devices 340 can be interconnected via system bus 350. Processor 310 is capable of processing instructions for execution within the computing system 300. Such executed instructions can implement one or more components of, for example, analyzer 1000, method 200 and/or any components therein. In some example embodiments, processor 310 can be a single-threaded processor. Alternately, processor 310 can be a multi-threaded processor. Processor 310 is capable of processing instructions stored in memory 320 and/or on the storage device 330 to display graphical information for a user interface provided via the input/output device 340.

Memory 320 is a computer readable medium such as volatile or non-volatile that stores information within computing system 300. Memory 320 can store data structures representing configuration object databases, for example. Storage device 330 is capable of providing persistent storage for computing system 300. Storage device 330 can be a floppy disk device, a hard disk device, an optical disk device, or a tape device, or other suitable persistent storage means. Input/output device 340 provides input/output operations for the computing system 300. In some example embodiments, input/output device 340 includes a keyboard and/or pointing device. In various implementations, the input/output device 340 includes a display unit for displaying graphical user interfaces.

According to some example embodiments, input/output device 340 can provide input/output operations for a network device. For example, input/output device 340 can include Ethernet ports or other networking ports to communicate with one or more wired and/or wireless networks (e.g., a local area network (LAN), a wide area network (WAN), the Internet).

In some example embodiments, computing system 300 can be used to execute various interactive computer software applications that can be used for organization, analysis and/or storage of data in various formats. Alternatively, computing system 300 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via input/output device 340. The user interface can be generated and presented to a user by computing system 300 (e.g., on a computer screen monitor, etc.).

One or more aspects or features of the subject matter described herein can be realized in digital electronic circuitry, integrated circuitry, specially designed application-specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

Embodiments of the present invention will be further described in the following examples, which do not limit the scope of the invention described in the claims.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A method comprising:
obtaining all raw melt curves for an assay plate from a memory;
determining melting peak curves for all standards and unknown samples;
calculating a median or mean peak curve for each of the standards based on a plurality of replicate melting peak curves of each standard;
calculating at least one correlation coefficient between each of the unknown samples and the median standards;
comparing the correlation coefficient to a threshold level for each standard; and
assigning the unknown sample a genotype of the standard when the unknown sample has a correlation coefficient greater than the threshold level.

2. The method of claim 1, further comprising assigning the unknown sample as an unknown genotype when the unknown sample has a correlation coefficient less than or equal to the threshold level.

3. The method of claim 2, wherein the unknown sample is assigned as an unknown genotype when it qualifies as a negative curve.

4. The method of claim 1, wherein determining the melting peak curves comprises determining first and second derivatives of each of the raw melt curves curve using a smoothing filter.

5. The method of claim 4, wherein the smoothing filter is a Savitzky-Golay technique.

6. The method of claim 1, wherein the melting peak curves are based on a plurality of parameters.

7. The method of claim 6, wherein the plurality of parameters include a channel number of the curve, temperature values of an input curve, fluorescence values of the input curve, a standard identification of the curve, and a melting temperature of the curve.

8. The method of claim 1, further comprising determining a standard curve quality metric to indicate how close each curve within a standard is to a curve representing the median or mean of that standard.

9. A system comprising:
a memory; and
a processor coupled to the memory and configured to:
obtain all raw melt curves for an assay plate from a memory;
determine melting peak curves for all standards and unknown samples;
calculate a median or mean peak curve for each of the standards based on a plurality of replicate melting peak curves of each standard;
calculate at least one correlation coefficient between each of the unknown samples and the median standards;
compare the correlation coefficient to a threshold level for each standard; and
assign the unknown sample a genotype of the standard when the unknown sample has a correlation coefficient greater than the threshold level.
